(19) United States
(12) Patent Application Publication
(10) Pub. No.: US 2026/0102619 A1
(43) Pub. Date: Apr. 16, 2026

(54) ANALYSIS OF COMPLEX ELECTROPHYSIOLOGICAL RESPONSES TO NEURAL STIMULATION OF MIXED NERVES

(71) Applicant: SALUDA MEDICAL PTY LIMITED, Macquarie Park (AU)

(72) Inventors: Leonardo Silvestri, Macquarie Park (AU); Daniel John Parker, Macquarie Park (AU); Peter Maxwell Ka-Shin Kiemann, Macquarie Park (AU)

(21) Appl. No.: 19/355,820

(22) Filed: Oct. 10, 2025

(30) Foreign Application Priority Data
Oct. 11, 2024 (AU) 2024903285
Dec. 23, 2024 (AU) 2024904279

(51) Int. Cl. A61N 1/36; A61N 1/02; G16H 20/40; G16H 40/67

(52) U.S. Cl. CPC A61N 1/36139; A61N 1/025; G16H 20/40; G16H 40/67

(57) ABSTRACT

Disclosed is a neural stimulation system and method. A neural stimulus is delivered according to a stimulus intensity parameter; and a signal window is captured from a signal sensed on neural tissue by a recording electrode subsequent to the neural stimulus. A plurality of signal windows are captured from signals sensed on the neural tissue by a first recording electrode subsequent to respective neural stimuli delivered according to respective stimulus intensity parameter values. Repeatedly, for each candidate detector in a set of candidate detectors: the candidate detector is used to measure intensities of evoked neural responses in the captured signal windows; and derive a metric for the candidate detector from the measured neural response intensities; and select a candidate detector for the first recording electrode from the set of candidate detectors based on their respective metrics.

The present application claims priority from Australian Provisional Patent Applications Nos. 2024903285 filed on 11 Oct. 2024 and 2024904279 filed on 23 Dec. 2024, the contents of each of which are incorporated herein by reference in their entirety.

### TECHNICAL FIELD

The present invention relates to neural stimulation and in particular to analysis of complex electrophysiological responses to neural stimulation of mixed nerves.

### BACKGROUND OF THE INVENTION

There are a range of situations in which it is desirable to apply neural stimuli in order to alter neural function, a process known as neuromodulation. For example, neuromodulation is used to treat a variety of disorders including chronic pain, movement disorders, and voiding disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal). Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), cither of which can significantly alter the neural recruitment arising from a given stimulus, and therefore the therapeutic range. The spinal cord itself moves within the cerebrospinal fluid (CSF) with respect to the dura and the electrode array. During postural changes, the amount of CSF or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

Applying neural stimulation therapy to voiding disorders is well known in the field. Voiding disorders include disorders that are influenced by sacral nerves. For example, disorders such as urinary incontinence, urinary urge/frequency, urinary retention, pelvic pain, bowel dysfunction (constipation, diarrhea), and sexual dysfunction are disorders influenced by the sacral nerves.

There are many differences between spinal cord stimulation (SCS) and sacral nerve stimulation (SNS). Firstly, the anatomy is different in the spinal cord and the pelvic floor. Secondly, the dimension and the physical properties of leads differ significantly between the two modalities. Further, the target nerve in SNS is a mixed nerve while the target neural pathway in SCS, the dorsal column, is predominantly composed of Aβ fibres. Therefore, rather than having one fibre type (Aβ fibres), in SNS, a multitude of fibre types will be activated by stimulation. Mixed nerves produce a complex electrophysiological response when stimulated. Components of an electrophysiological response to SNS may be:
Evoked neural responses of different fibre types (Aα, Aβ, Aγ, Aδ, B, C, . . . ). These can be “early” (referred to herein as evoked compound action potentials or ECAPs, which propagate along fibres) or “late” (referred to herein as “late responses”, which do not propagate along fibres but essentially appear simultaneously at all locations in the tissue).
Myoclectric responses (referred to as “EMGs”) representing a muscular contraction. These responses do not propagate along nerves but are mid/far-field recordings of the electrical effects of muscular contraction that appear substantially simultaneously at all locations in the tissue. Myoclectric responses, although they do not themselves propagate, are activated by a propagating Aα efferent ECAP arriving at a certain location in the contracting muscular tissue.

It is, therefore, important to determine the type(s) of fibres recruited due to the stimulation of the sacral nerve, and how the degree of recruitment of each type varies with intensity, in order to provide the best outcome for the patient. In particular, stimulating certain fibre types could result in unpleasant side effects in the patient.

However, determining the components of an electrophysiological response is conventionally manual and difficult. Engineers/clinicians visually inspect signals obtained from different measurement and stimulus parameters and try to determine whether the electrophysiological response is of neural origin, myoelectric, or a mix of them. This is time consuming, requires a lot of training, and is limited by the natural ability of humans to detect patterns and interpret multi-dimensional relationships.

Any discussion of documents, acts, materials, devices, articles or the like which has been included in the present Background is solely for the purpose of providing a context for the present technology. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide methods and devices for automatically measuring an evoked therapeutic component of a complex electrophysiological response of a mixed nerve to neural stimulation, which will overcome or substantially ameliorate at least some of the deficiencies of the prior art, or at least provide an alternative.

Some implementations herein relate to a neural stimulation system. For example, the neural stimulation system may include an implantable device for controllably delivering neural stimuli, the device may include: a stimulus source configured to deliver neural stimuli via one or more stimulus electrodes of a plurality of implanted electrodes to a neural pathway of a patient in order to evoke neural responses from the neural tissue; measurement circuitry configured to capture signal windows from signals sensed on the neural tissue subsequent to respective neural stimuli by a recording electrode of the plurality of implanted electrodes; and a control unit configured to: control the stimulus source to deliver a neural stimulus according to a stimulus intensity parameter; and control the measurement circuitry to capture a signal window from a signal sensed on the neural tissue by a recording electrode subsequent to the neural stimulus. The neural stimulation system may also include a processor configured to: instruct the control unit to control the measurement circuitry to capture a plurality of signal windows from signals sensed on the neural tissue by a first recording electrode subsequent to respective neural stimuli delivered according to respective stimulus intensity parameter values; repeatedly, for each candidate detector in a set of candidate detectors: measure, using the candidate detector, intensities of evoked neural responses in the captured signal windows; and derive a metric for the candidate detector from the measured neural response intensities. The processor may furthermore select a candidate detector for the first recording electrode from the set of candidate detectors based on their respective metrics.

Some implementations herein relate to an automated method. For example, the method may include delivering a plurality of neural stimuli to neural tissue of a patient in order to evoke neural responses from the neural tissue, the neural stimuli being delivered according to respective stimulus intensity parameter values; capturing a plurality of signal windows from signals sensed on the neural tissue by a first recording electrode subsequent to respective neural stimuli delivered according to respective stimulus intensity parameter values; repeatedly, for each candidate detector in a set of candidate detectors: measuring, using the candidate detector, intensities of evoked neural responses in the captured signal windows; and deriving a metric for the candidate detector from the measured neural response intensities. The method may furthermore include selecting a candidate detector for the first recording electrode from the set of candidate detectors based on their respective metrics.

The present invention has been developed primarily for use in or with stimulation of the sacral nerve and will be described hereinafter mostly with reference to this application. However, it will be appreciated that the present invention is not limited to this particular field of use, and may be applied in other neuromodulation contexts, including but not limited to spinal cord stimulation, pudendal nerve stimulation, deep brain stimulation, stimulation of other parts of the peripheral and central nervous system. It will further be appreciated that the present invention may be applied for treatment of conditions other than pelvic floor disorders, including but not limited to chronic pain, movement disorders, Crohn's disease, rheumatoid arthritis, diabetes, Reynaud's phenomenon, chronic inflammatory conditions, migraine, stroke, or depression.

### BRIEF DESCRIPTION OF THE DRAWINGS

Notwithstanding any other implementations which may fall within the scope of the present invention, one or more implementations of the present invention will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a bundle of target nerve fibres;

FIG. 4a contains a graph illustrating a piecewise-linear activation plot model for one posture of a patient undergoing neural stimulation;

FIG. 4b contains a graph illustrating an alternative activation plot model for one posture of a patient undergoing neural stimulation;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a block diagram illustrating the data flow of a neural stimulation therapy system such as the system of FIG. 7;

FIG. 9 is a schematic representation of the functional separation of fibres in the ventral rootlets of a human S2 nerve in cross-section;

FIG. 10 is a graph showing captured signals representing an electrophysiological response obtained from the S3 sacral nerves of a human patient undergoing SNS therapy;

FIG. 11 is a flow chart illustrating a method of obtaining an ensemble of signals representing a complex electrophysiological response of neural tissue containing a mixed nerve to neural stimulation captured under different capture conditions, according to one aspect of the present technology;

FIG. 12 is a graph showing an ensemble of differentially recorded signals captured in accordance with the method of FIG. 11;

FIG. 13 is a graph showing the response intensities measured from the ensemble of FIG. 12 plotted against the respective stimulus intensities;

FIG. 14 is a flow chart illustrating a method of deriving a customised vector detector for measuring an evoked therapeutic component of a complex electrophysiological response of a mixed nerve to neural stimulation, according to one aspect of the present technology;

FIG. 15 is a graph containing a subset of signals captured at a single recording electrode over a ramp of stimulus intensities;

FIG. 16a is a graph on which are plotted the first three basis functions obtained using the method of FIG. 14;

FIG. 16b is a graph showing the first three activation plots corresponding to the basis functions of FIG. 16a;

FIG. 17a is a graph on which is plotted the third basis function from FIG. 16a, transformed by rotation according to one aspect of the present technology; and

FIG. 17b is a graph containing the resulting activation plot, obtained by correlating the rotated basis function of FIG. 17 with the subset of signals illustrated in FIG. 15; and

FIG. 18 is a flow chart illustrating a method of deriving a set of customised vector detectors for measuring respective evoked therapeutic components of a complex electrophysiological response of a mixed nerve to neural stimulation, according to another aspect of the present technology.

### DETAILED DESCRIPTION OF THE PRESENT TECHNOLOGY

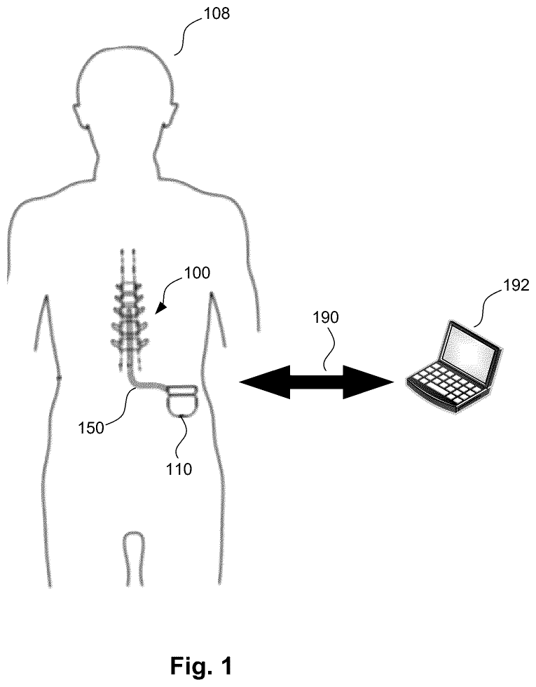

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

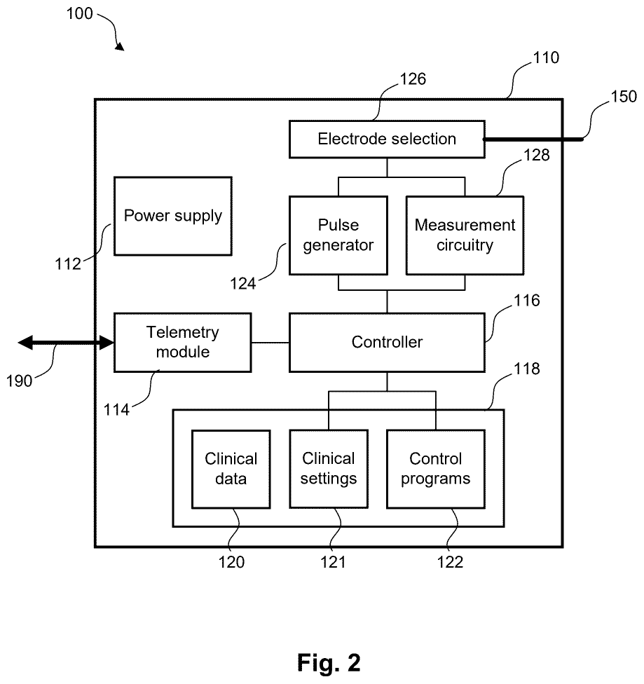

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode array 150 as selected by electrode selection module 126.

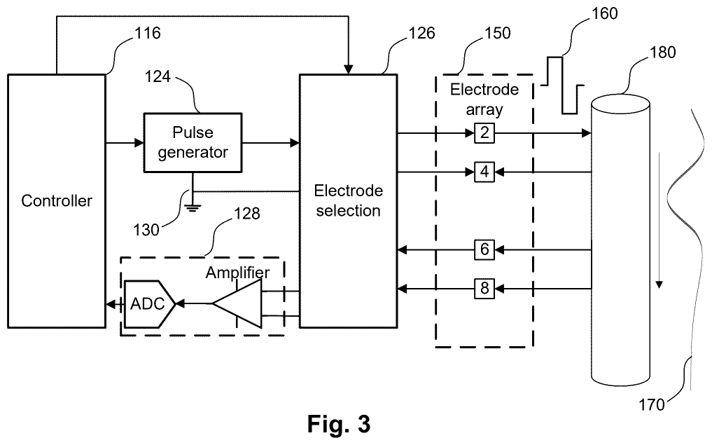

FIG. 3 is a schematic illustrating interaction of the implanted stimulator 100 with a bundle of target nerve fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve, or a brain structure. Electrode selection module 126 selects a stimulus electrode 2 of electrode array 150 through which to deliver a pulse from the pulse generator 124 to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150. The set of stimulus electrodes and return electrodes is referred to as the stimulus electrode configuration (SEC). Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be associated with paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

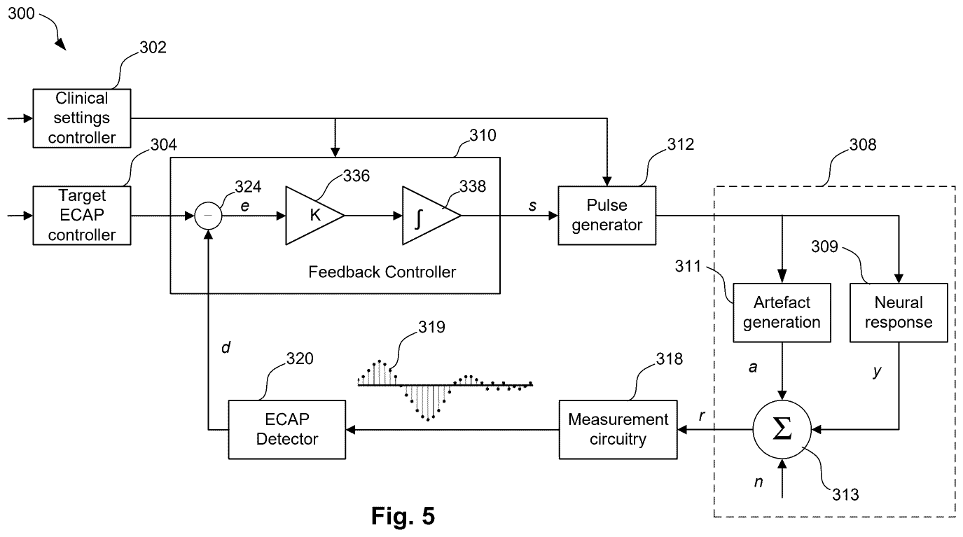

FIG. 6 illustrates the typical form of an ECAP 600 of a healthy subject, as sensed by a single measurement electrode referenced to the system ground 130 or referenced to an indifferent electrode. Such configurations are referred to as single-ended ECAP measurement. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along target fibres 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration (MEC). The measurement circuitry 128 for example may operate in accordance with the teachings of the above-mentioned International Patent Publication No. WO2012/155183.

Monopolar or single-ended measurement electrode configurations comprise an ‘indifferent’ reference electrode that senses an insubstantial amount of the evoked electrophysiological response. Techniques for selecting an indifferent reference electrode are disclosed in International Patent Publication no. WO2023/235926 by the present applicant, the contents of which are herein incorporated by reference. Single-ended measurement electrode configurations may sense single-ended responses as illustrated in FIG. 6.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response. The parameters of the ECAP detector, together with the measurement electrode configuration, make up the measurement parameters.

Stimulator 100 applies stimuli over a potentially long period such as days, weeks, or months and during this time may store characteristics of neural responses, clinical settings, target response intensity, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

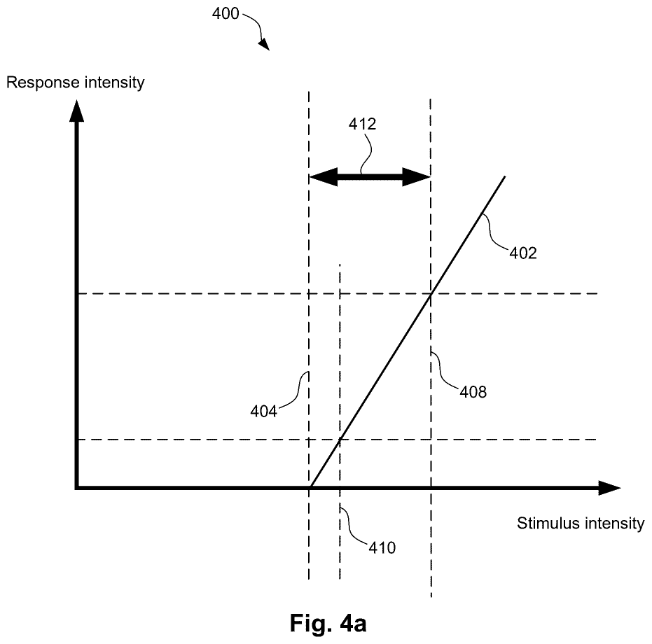

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a contains a graph 400 illustrating an activation plot model 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$d ( s ) = \{ \begin{matrix}S ( s - T ) , & s \geq T \\ 0 , & s < T\end{matrix} \qquad ( 1 )$$

where s is the stimulus intensity, d is the ECAP amplitude, T is the ECAP threshold and S is the slope of the activation plot (referred to herein as the patient sensitivity) above the ECAP threshold T. The sensitivity S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 is a value of stimulus intensity that corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

An alternative to piecewise linear model of equation (1) is the golden growth curve (GGC) model. The GGC model, like the piecewise linear model, is a continuous model comprising a sub-threshold linear portion of constant zero intensity and a supra-threshold linear portion. However, in the GGC model, these two portions are joined by a transitional portion of variable curvature. In one implementation, the GGC model is derived from a multi-parameter function g(x|τ, x₀) with two such linear portions and a curved transitional portion. The parameters of the template function g are:
x₀, the x-intercept of the supra-threshold linear portion and therefore the approximate location (the knee point) of the transitional portion;
τ, a curvature parameter that sets the curvature of the transitional portion (the smaller the value of curvature t, the sharper the transition).

One implementation of the GGC model is the difference between two different versions of the template function g, with the two versions of g having different transitional locations and curvatures but the same scaling:

$$d ( s ) = {Ps}_{T} [ g ( \frac{s}{{s}_{T}} \vert {τ}_{1 ,} 1 ) - g ( \frac{s}{{s}_{T}} \vert {τ}_{2} , r ) ] \qquad ( 2 )$$

This implementation of the GGC model comprises three distinct portions: a sub-threshold portion of zero intensity, a supra-threshold linear portion joined to the sub-threshold portion by a first transitional portion around an x-intercept s_{T}, and a saturation portion that approaches a saturation value joined to the supra-threshold portion by a second transitional portion around a saturation threshold. The parameters of such an implementation of the GGC model are:
P, the slope of the supra-threshold linear portion, related to the patient sensitivity S.
s_{T}, the x-intercept of the supra-threshold linear portion, related to the ECAP threshold
τ₁, the curvature parameter of the first transitional portion.
τ₂, the curvature parameter of the second transitional portion
r, the ratio of the saturation threshold to the intercept s_{T}.

In other implementations, a more general GGC model comprising further parameters may be used. For example, a GGC model may comprise a sub-threshold portion with non-zero intensity such as a constant intensity (one further parameter) or a linear profile (two further parameters), to model the effect of any artefact that leaks through the ECAP detector.

FIG. 4b contains a graph 450 illustrating a GGC model 460. It may be seen that the GGC 460 saturates at higher stimulus intensities. The vertical line 480 represents the intercept s_{T} and the vertical line 490 represents the saturation threshold (the saturation ratio r times the intercept s_{T}). The therapeutic range 475 of the GGC is the range of stimulus intensity values between the intercept s_{T} and the saturation threshold r×s_{T}.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at or near a target response intensity. For example, the device may calculate an error between a target ECAP amplitude and a measured ECAP amplitude, and adjust the applied stimulus intensity to bring the measured ECAP amplitude closer to the target ECAP amplitude, such as by adding the scaled error to the current stimulus intensity. A neuromodulation device that operates by adjusting the applied stimulus intensity to maintain a feedback variable at or near a target value is said to be operating in closed-loop mode and will also be referred to as a closed-loop neural stimulation (CLNS) device. By adjusting the applied stimulus intensity to maintain the measured ECAP amplitude at or near an appropriate target response intensity, a CLNS device will generally keep the stimulus intensity within the therapeutic range as patient posture varies.

A CLNS device comprises a pulse generator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, may be controlled by the feedback loop.

In an example CLNS system, the user sets a target response intensity, and the CLNS device performs proportional-integral-differential (PID) control. In some implementations, the differential contribution is disregarded and the CLNS device uses a first order integrating feedback loop. The pulse generator produces a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

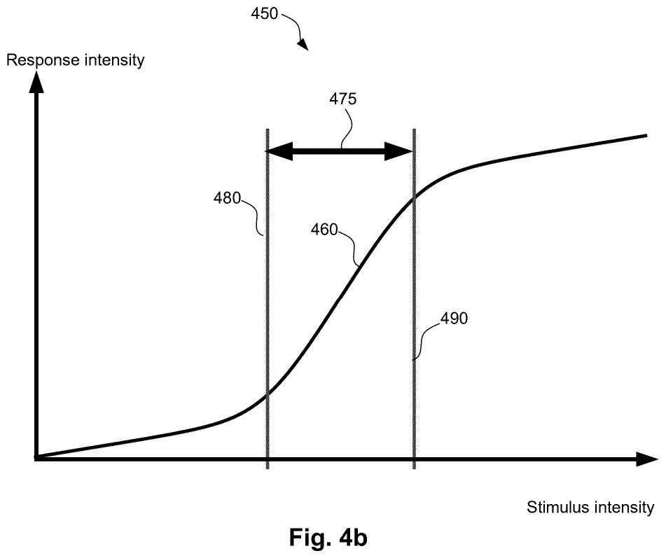

FIG. 5 is a schematic illustrating elements and inputs of a closed-loop neural stimulation (CLNS) system 300, according to one implementation of the present technology. The system 300 comprises a pulse generator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the stimulus electrode configuration (including the number of stimulus electrode poles), the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal r to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int K edt \qquad ( 3 )$$

where K is the gain of the gain element 336 (the controller gain). This relation may also be represented as

$$δ s = Ke \qquad ( 4 )$$

where δs is an Adjustment to the Current Stimulus Intensity Parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the pulse generator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the pulse generator 312 generates a stimulus in accordance with the adjusted stimulus intensity s. Accordingly, there is a delay of one stimulus clock cycle before the stimulus intensity is updated in light of the error value e.

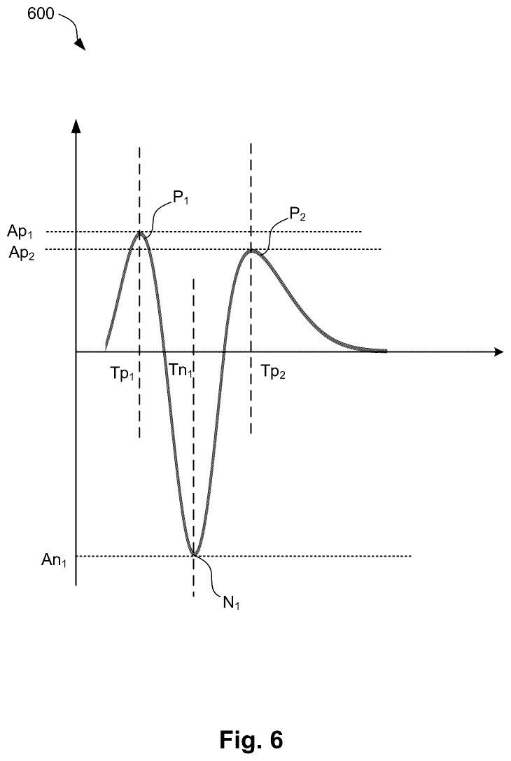

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Data Flow

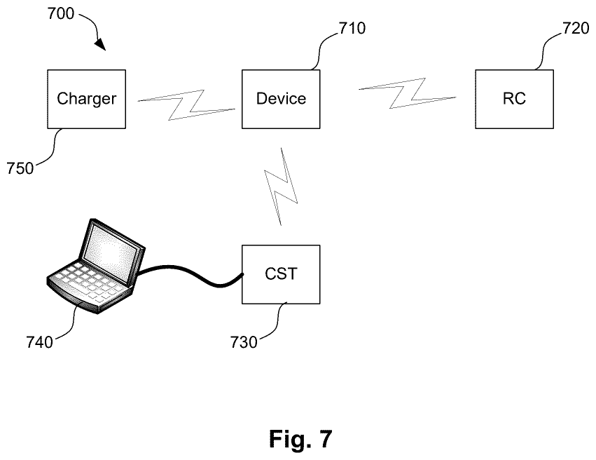

FIG. 8 is a block diagram illustrating the data flow 800 of a neural stimulation therapy system such as the system 700 of FIG. 7 according to one implementation of the present technology. Neuromodulation device 804, once implanted within a patient, applies stimuli over a potentially long period such as weeks or months and records neural responses, clinical settings, target response intensity, and other operational parameters, discussed further below. Neuromodulation device 804 may comprise a Closed-Loop Neural Stimulation (CLNS) device, in that the recorded neural responses are used in a feedback arrangement to control clinical settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining sensed signals subsequent to every stimulus, or at least obtaining such sensed signals regularly. Each sensed signal generates a feedback variable such as a measure of the amplitude of the evoked neural response, which in turn results in the feedback loop changing at least one stimulus parameter for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session; (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service which runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web API 826 which provides data for third-party analysis such as by the analysis module 832, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### Mixed Nerve Responses

Almost all major nerves in the periphery are of mixed nature, meaning that the nerve contains fibres of various types and functions that run together. The peripheral nerves bundle together at various stages and form the spinal nerves (such as the S3 sacral nerve, which is the main target for SNS). Before joining the spinal cord, the spinal nerves split up into the ventral and dorsal roots. In simplified terms, the ventral roots contain mostly a variety of efferent fibres, and the dorsal roots contain mostly a variety of afferent fibres. Mixed nerves therefore can contain both afferent and efferent fibre types. Mixed nerves are heterogenous collections of fascicles, and it has been shown that the fascicles bundle nerve fibres that serve similar functions and share common physiological properties. This separation of function can be observed from the rootlets which form the dorsal and ventral roots of each spinal nerve.

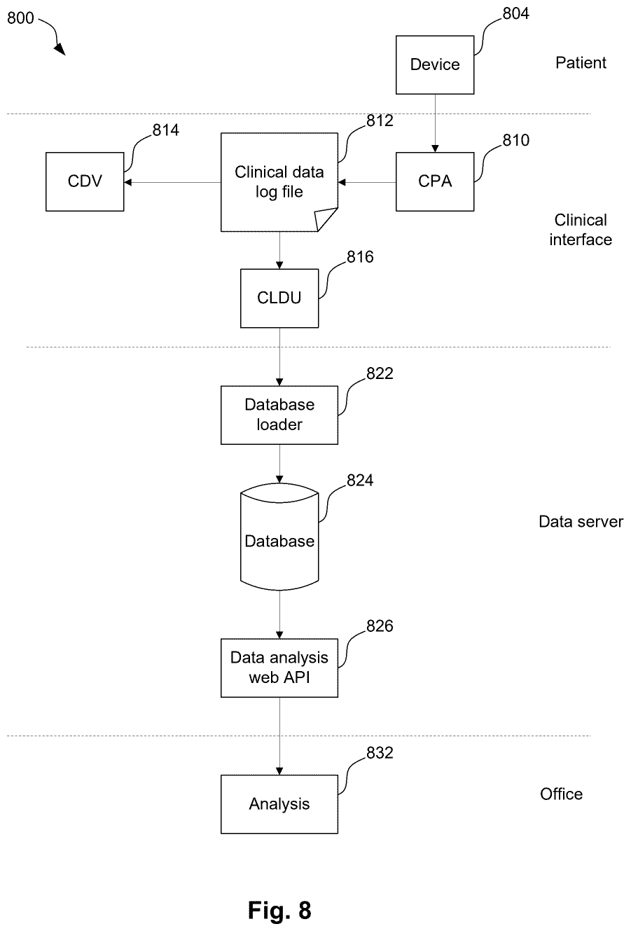

FIG. 9 is a schematic representation of the functional separation of fibres in the ventral root of a human S2 nerve in cross-section. The root consists of 2 rootlets. Three different nerve distribution patterns arise: the somatic type (labelled S) with predominantly large, thickly myelinated fibres and an absence of parasympathetic fibres; the vegetative type (labelled V) with an abundance of parasympathetic fibres; and the mixed type (labelled M). Note the topographic aggregation of the fascicles of vegetative and somatic types. The vegetative type fascicles with a predominance of parasympathetic fibres are concentrated in the right rootlet of the ventral root. In contrast, purely somatic fascicles are found in the left rootlet. It appears that the nerve fibres do not simply follow a random distribution, but rather some sort of functional organization.

Stimulation of any given subsection of a mixed nerve, for example by applying stimuli only from one side of the nerve at an amplitude which only recruits fibres in fascicles proximal to the stimulus electrode, will therefore activate a particular portion of fibres that serve a distinct function. The present technology recognises that targeting the appropriate fibres of a mixed nerve is of great importance for many neuromodulation applications, including SNS.

However, such targeting is in essence impossible using a purely anatomical approach, because the course of each fascicle in a mixed nerve varies along the nerve. The fascicles can cross, merge, and split, so that a desired fibre type takes significantly varying positions within the nerve at different parts of the nerve. Further, inter-patient variability of the content and disposition of fascicles within a nerve exists. These changes occur on scales which are significantly smaller than a typical stimulus electrode spacing, as typical implanted electrode arrays utilise electrodes which are 3 mm long, and arc 4 mm apart from each other, i.e. positioned on a 7 mm pitch. However, in the space of 7 mm along a nerve, any given nerve fascicle could take any or all positions within the bundle making it impossible to effectively selectively target that fascicle if based only on surrounding anatomical orientations. Simplistically utilising smaller electrodes would not resolve these uncertainties.

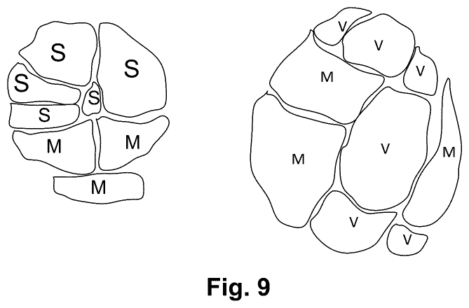

FIG. 10 is a graph showing captured signals representing an electrophysiological response obtained from the S3 sacral nerves of a human patient undergoing SNS therapy. The responses of FIG. 10 were obtained by stimulating from standard cylindrical (ring) electrodes, therefore preferentially activating the fibres on the side of the nerve which was in closest proximity to the electrode. In FIG. 10 each of the three signals were sensed by respective different single-ended measurement electrode configurations. The measurement electrode configurations respectively comprise consecutive recording electrodes along the lead with increasing distance from the stimulus electrode on the same lead (6 mm, 12 mm and 18 mm from the stimulus electrode, respectively) and a common indifferent reference electrode, so that neural responses propagating as action potentials along a nerve occur later in time on the signals from more distant recording electrodes. By contrast, non-propagating components are not spaced apart in time across the respective signals, allowing neural responses to be distinguished from other electrophysiological activity. In FIG. 10, electrode 4 was used for stimulation, so that the signal 1010 (labelled as CH3) was recorded closest to the stimulus electrode and the signal 1020 labelled as CHI was recorded furthest from the stimulus electrode.

In FIG. 10 an Aβ ECAP may be observed in the timeframe around 1 to 2 ms, and a myoelectric response between 4 and 10 ms. The ECAP in the timeframe around 1 to 2 ms may be specifically identified as an Aβ ECAP because of the finite conduction velocity and the distance of each recording electrode from the stimulus site. Further, the component observed in the timeframe around 4 to 10 ms is non-propagating, as evidenced by simultaneous appearance of this component on all three electrodes. While the fast conduction velocity of the Aβ ECAP means that this component is obscured by artefact in the very early part of the signals (e.g. in the period<1 ms), the existence of an Aa (A-alpha) efferent response component may be inferred because of the existence of the non-propagating signal component (labelled “EMG”) observed in the timeframe around 4 to 10 ms. The EMG is an electrical field resulting from muscle activation and which therefore must arise due to Aa efferent activation (either from direct activation or via a reflex arc by means of, for example, la afferent fibre activation) as muscle activation is the role of Aa efferent fibres.

### Analysis of Mixed Nerve Responses

Dot product (correlation) ECAP detectors are robust to noise and have proven effective in measuring the intensity of neural responses evoked in Aβ fibres in the dorsal column, which are therapeutic for pain relief. Such correlation detectors comprise a vector that roughly matches the morphology of the evoked neural response. The vector may be derived from a predetermined template such as the four-lobe filter disclosed in the above-mentioned International Patent Publication No. WO2015/074121, with time scale and offset customised to the particular patient and capture conditions. The derivation of the vector detector from a predetermined template, with customisation of time scale and offset, is based on the presumption that the neural response has a reasonably consistent morphology between patients and capture conditions. In particular, the presumption is that the morphology remains reasonably consistent as stimulus intensity varies over the therapeutic range. The four-lobe filter template was derived from observations of neural responses to spinal cord stimulation that generally met this presumption. However, such a template is not necessarily suitable for measuring the components of complex electrophysiological responses such as obtained from sacral nerve stimulation.

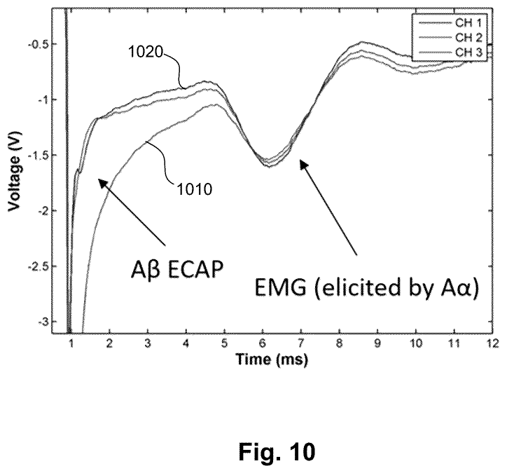

FIG. 11 is a flow chart illustrating a method 1100 of obtaining an ensemble of signals representing a complex electrophysiological response of neural tissue containing a mixed nerve to neural stimulation captured under different capture conditions, according to one aspect of the present technology. The method 1100 may be carried out by an external device, such as the clinical interface 740, in communication with a neuromodulation device such as the device 710. The clinical interface 740 may be configured to implement the method 1100 by a suitable Clinical Programming Application (CPA) stored in an instruction memory of the clinical interface. Alternatively, the method 1100 may be carried out by a controller such as the controller 116 of the electronics module 110 of the stimulator 100. The controller 116 may be configured to implement the method 1100 by firmware 122 stored in the memory 118 of the electronics module.

The method 1100 starts at step 1110, which chooses new signal capture conditions. The signal capture conditions for each iteration of the steps 1120 to 1140 comprise the stimulus parameters and the measurement parameters. In one example, step 1110 chooses a new measurement electrode configuration while keeping the same stimulus parameters. In another example, step 1110 chooses a new stimulus intensity parameter value while keeping the other stimulus parameters, and the measurement parameters, the same.

The next step 1120 instructs the pulse generator 124 to deliver a stimulus to the neural tissue according to the stimulus parameters of the chosen capture conditions. Step 1130 then instructs the measurement circuitry 128 to capture a signal window subsequent to the delivered stimulus, and representing the electrophysiological response of the neural tissue thereto, according to the measurement parameters of the chosen capture conditions. Step 1140 then adds the captured signal window to an ensemble of signal windows (also referred to herein as signals) representing the electrophysiological response.

Step 1150 then checks whether there are any more conditions under which a signal window is to be captured. If so (“Y”), the method 1100 returns to step 1110 to choose new capture conditions. Otherwise (“N”), the method 1100 ends at step 1160.

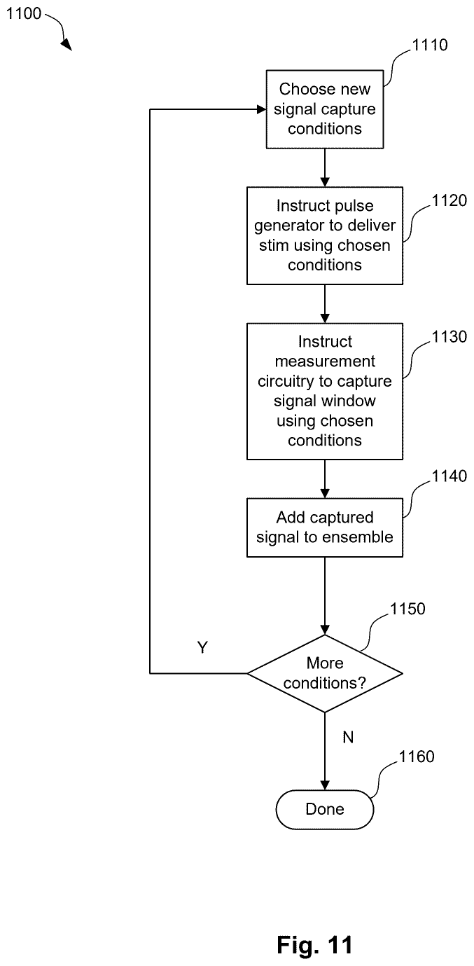

In some implementations, the method 1100 may not be implemented as a sequential loop over repeated stimuli delivered according to different stimulus parameters, but may instead capture multiple signal windows near-simultaneously according to different capture conditions subsequent to a single delivered stimulus. Such implementations are suitable for the case where the different capture conditions comprise different measurement parameters and the same stimulus parameters, as illustrated for example in FIG. 10. In other implementations, the method 1100 may be implemented as a sequential loop over repeated stimuli delivered according to different stimulus parameters, with responses captured at a single measurement electrode configuration, as illustrated for example in FIG. 12. FIG. 12 is a graph showing an ensemble 1200 of differentially recorded signals, such as the signal 1210, captured in accordance with the method 1100. The ensemble 1200 represents complex electrophysiological responses to a set of respective sacral nerve stimuli of increasing stimulus intensities captured at a single MEC. There is no clearly discernible component of consistent morphology with an intensity that increases as stimulus intensity increases within the ensemble 1200. In particular, correlation of the signals in the ensemble 1200 with a vector detector derived from the four-lobe filter does not result in a well-behaved activation plot.

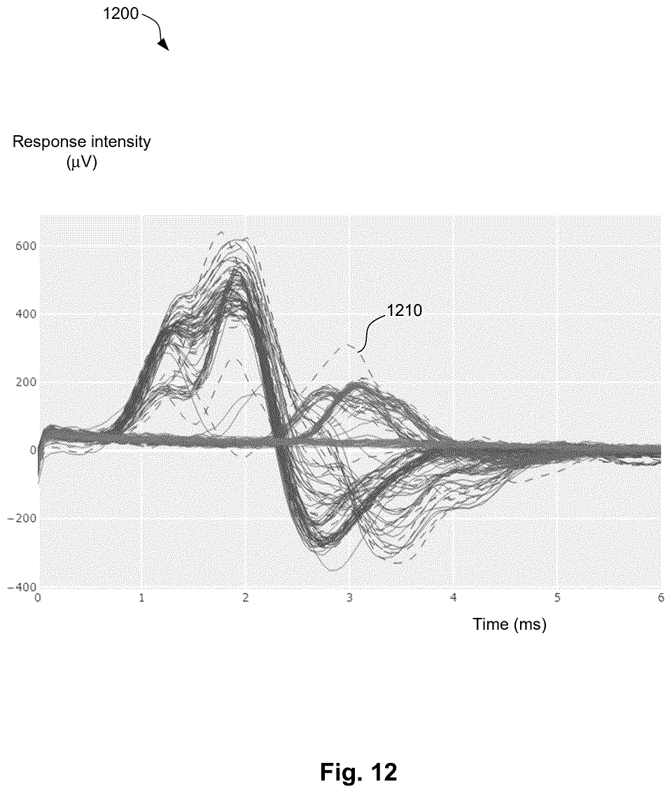

FIG. 13 is a graph 1300 showing the response intensities measured from the ensemble 1200 using a vector detector derived from the four-lobe filter, with optimally tuned time scale and delay, plotted against the respective stimulus intensities. It may be seen that the response intensity measurements in the graph 1300 do not form an activation plot of the piecewise linear form illustrated in FIG. 4a. This illustrates that a four-lobe filter template is not necessarily suitable for measuring the components of complex electrophysiological responses such as obtained from sacral nerve stimulation.

Methods and systems according to one aspect of the present technology are configured to derive a customised vector detector from an ensemble of signals captured subsequent to test stimuli over a range of stimulus intensities at one or more different MECs. In the methods and systems according to the present technology, a set of candidate detectors is constructed from a subset of the ensemble consisting of signals captured at a single MEC. Each candidate detector is applied to the subset to obtain a set of response intensities similar to the set illustrated in FIG. 13. A metric is determined from each candidate detector based on the set of response intensities. The candidate detector with the highest value of the metric is selected as the customised vector detector for the MEC corresponding to the subset. The customised detector may then be applied to signals captured subsequent to therapeutic stimuli delivered under the same conditions, to measure the intensities of the evoked therapeutic components. Such response intensity measurements may be used for efficacy monitoring and possibly closed-loop adjustments to stimulus parameters such as stimulus intensity to maintain response intensity at or near a target value, as described above in relation to FIG. 5.

The metric is configured to select a customised detector with the best combination of controllability and physiologicality for the particular patient and SEC/MEC combination. Controllability refers to the “goodness of fit” of the plot of response intensities versus stimulus intensities to an activation plot model such as the piecewise linear model illustrated in FIG. 4a. Controllability is an indicator of the usefulness of a given candidate detector to provide a feedback variable for a feedback controller of a CLNS system. Physiologicality refers to the closeness of a match between the fitted activation plot model and the sensations experienced by the patient during the test stimuli. In particular, a candidate detector will receive a high physiologicality score to the extent that its fitted activation plot parameters match the patient's subjective perceptual markers such as perception threshold and discomfort threshold. The presumption underlying the present technology is that an evoked therapeutic component of the complex electrophysiological response of a mixed nerve such as the sacral nerve behaves similarly to the evoked neural response to spinal cord stimulation, which is known to be indicative of efficacy in treating chronic pain. Therefore, a vector detector customised to extract measurements from responses exhibiting such behaviour is likely to provide useful information for therapeutic stimulation of mixed nerves.

### Customisation of Vector Detectors

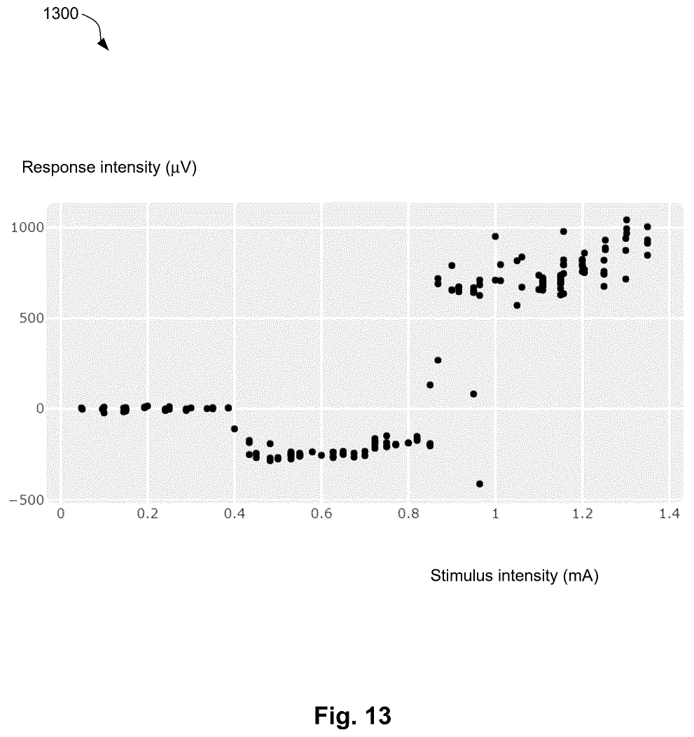

FIG. 14 is a flow chart illustrating a method 1400 of deriving a customised vector detector for measuring an evoked therapeutic component of a complex electrophysiological response of a mixed nerve to neural stimulation, according to one aspect of the present technology. The method 1400 may be carried out by an external device, such as the clinical interface 740, in communication with a neuromodulation device such as the device 710. The clinical interface 740 may be configured to implement the method 1400 by a suitable Clinical Programming Application (CPA) stored in an instruction memory of the clinical interface. Alternatively, the method 1400 may be carried out by a controller such as the controller 116 of the electronics module 110 of the stimulator 100. The controller 116 may be configured to implement the method 1400 by firmware 122 stored in the memory 118 of the electronics module.

The method 1400 starts at step 1410, which captures an ensemble of signals representing the complex electrophysiological response to test stimuli delivered under different capture conditions, with the patient in a fixed posture. The different capture conditions in step 1410 comprise a constant SEC, at a location predetermined as providing effective therapy while the patient is in the fixed posture. The capture conditions vary across two dimensions: the recording electrode distance from the SEC, and the stimulus intensity parameter. The reference electrode may be a predetermined, indifferent electrode so that the recordings are single-ended. The result of step 1410 is an ensemble of signals at different recording electrodes and stimulus intensities. Step 1410 may be implemented using the method 1100.

Step 1410 may, during the capturing of the ensemble of signals, capture perceptual markers such as perception threshold sₚ and discomfort threshold sa at each stimulus intensity and store these perceptual markers in association with the ensemble. In one implementation, the stimulus intensity parameter is gradually and discretely ramped upwards from zero at a predetermined ramp rate, and the signals at all recording electrodes are captured simultaneously after each discrete increase in the stimulus intensity. The patient is asked to indicate when they start to perceive a sensation of stimulation, and the stimulus intensity parameter value at that first indication of perception is recorded as the perception threshold sₚ. The ramp is continued, and the patient is the asked to indicate when they start to feel discomfort from the stimulation. The stimulus intensity parameter value at that first indication of discomfort is recorded as the discomfort threshold sa.

The next step 1420 extracts a subset of the ensemble comprising signals captured at a single, predetermined recording electrode (with varying stimulus intensities). Step 1420 then constructs a set of candidate detectors from the subset.

In a first implementation, step 1420 uses the singular value decomposition (SVD) of a data matrix X whose columns are the signals from the extracted subset. If there are m signals in the subset, i.e. m signals captured at different stimulus intensities at the predetermined recording electrode, and each signal comprises N samples, the matrix X is N rows by m columns. The SVD of the data matrix X is a factorisation into three matrices U, Σ, and V^{T}:

$$X = U \sum {V}^{T} \qquad ( 5 )$$

where the matrices U (which is N rows by m columns) and V (which is m by m) are unitary matrices (i.e. their transposes are their inverses) and Σ is an m by m diagonal matrix whose diagonal entries are the singular values σᵢ of the data matrix X, by convention in descending order of magnitude.

The columns of the matrix U may be interpreted as a set of m orthonormal basis functions for the space spanned by the columns of the data matrix X, i.e. the signals in the subset. The m singular values represent the relative importance of the respective basis functions in approximating all the signals in the subset.

According to the first implementation of step 1420, the first p of the m basis functions (i.e. the first p columns of U, corresponding to the p largest singular values σᵢ) form the kernel of the set of candidate detectors. The number p of candidate detectors in the kernel may be set to a small predetermined integer such as 3, as experience has shown that the remaining m-p singular values contribute very little to the explanatory power of the kernel when p is equal to 3.

Alternatively, the number p of candidate detectors in the kernel may be dynamically chosen based on the explanatory power of the m basis functions. In one such implementation, p is chosen to be the number of basis functions whose combined explanatory power exceeds a threshold, such as 95%. The explanatory power of a set of p basis functions may be obtained from the sum of the squares of the p largest singular values σᵢ in the matrix Σ, as a fraction of the total sum of the squares of all m singular values. The resulting value of p is roughly equivalent to the number of significant independent components in the subset of signals, including stimulus artefact.

The resulting p basis functions and their corresponding singular values and weights may be used to form an approximation \tilde{X} to the data matrix X.

$$\tilde{X} = {U}_{p} {\sum }_{p} {V}_{p}^{T} \qquad ( 6 )$$

where Uₚ is the first p columns of U, Vₚ is the first p columns of V, and Σₚ is a diagonal matrix whose diagonal entries are the p largest singular values of the data matrix X.

It may be shown using the properties of the SVD that \tilde{X} is the best possible approximation (in a least-squares sense) to the data matrix X that may be formed with p basis functions.

In addition, the i-th column of Vₚ (where i=1, . . . , p), scaled by the i-th singular value σᵢ, is the set of dot products (correlations) of the corresponding basis function (the i-th column of Uₚ) with the m signals in the subset. Plotting each scaled column of Vₚ against stimulus intensity gives the activation plot that would result if the corresponding basis function were used as the vector detector on the signals making up the data matrix X.

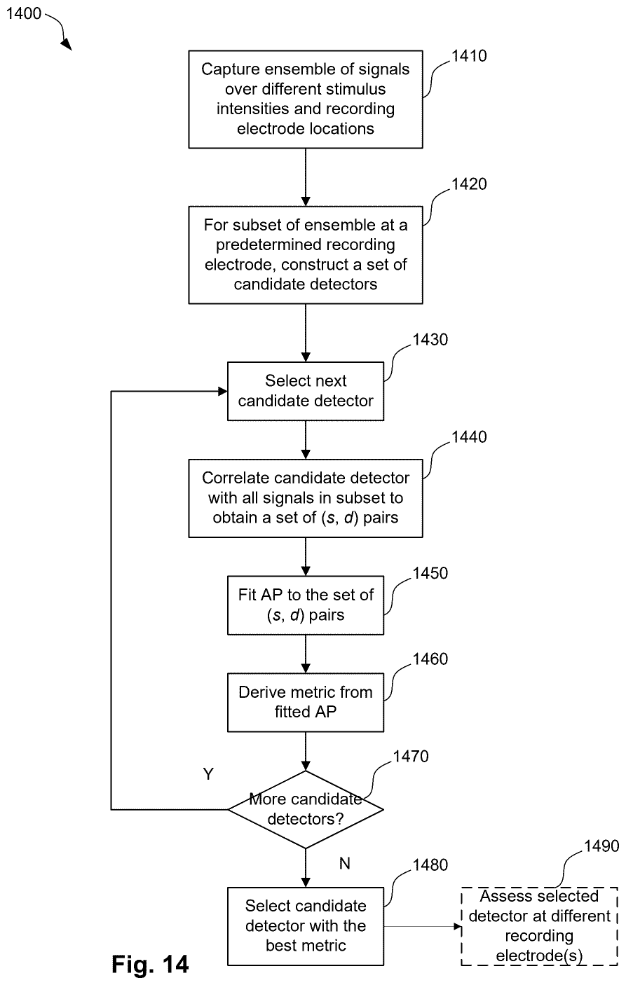

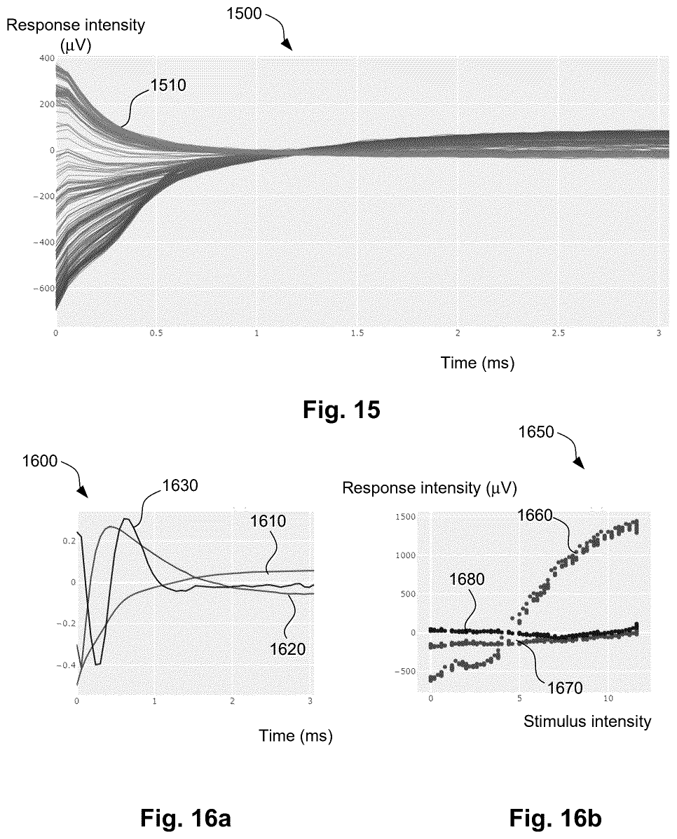

FIG. 15 is a graph 1500 containing such a subset of signals, e.g. the signal 1510, captured at a single recording electrode over a ramp of m stimulus intensities. FIG. 16a is a graph 1600 on which are plotted the first three basis functions, labelled 1610, 1620, and 1630 respectively, obtained using the SVD as described above with p=3. FIG. 16b is a graph 1650 showing the first three columns of V, scaled by the respective singular values, plotted against stimulus intensity. The resulting three activation plots (which correspond to the three basis functions 1610, 1620, and 1630 respectively) are labelled 1660, 1670, and 1680 respectively. None of the three activation plots 1660, 1670, and 1680 shows a marked resemblance to either of the physiologically-based models of FIGS. 4a and 4b, indicating that none of the three basis functions 1610, 1620, or 1630 would be particularly suitable as a choice of vector detector to be applied to the signals in the graph 1500 if we expect to obtain measurements of an evoked therapeutic component of the response.

To augment the set of candidate basis functions, the first implementation of step 1420 may apply a set of transformations to the kernel of p basis functions. For example, step 1420 may augment the kernel of p basis functions by multiplying Uₚ by a set {T} of orthonormal p-by-p matrices T. In one such implementation, suitable for the case where p is 3, each transformation matrix T is a 3-by-3 rotation matrix T(θ) where θ is a vector of three angles of rotation around respective axes. If the vector θ consists of angles α, β, and γ, of rotation about the z-, y-, and x-axes respectively, it may be shown that the rotation matrix T(θ) may be determined as

$$T ( θ ) = [ \begin{matrix}cos αcosβ & cos αsinβsinγ - sin αcosγ & cos αsinβcosγ + sin αsinγ \\ sin αcosβ & sin αsinβsinγ + cos αcosγ & sin αsinβcosγ - cos αsinγ \\ - sin β & cos βsinγ & cos βcosγ\end{matrix} ] \qquad ( 7 )$$

Note that if all three angles α, β, and γ are zero, the rotation matrix is the 3-by-3 identity matrix, so U₃T(0)=U₃.

The result is a set of candidate detectors {U₃T(θ)} where each component of the vector θ varies between 0 and 360°.

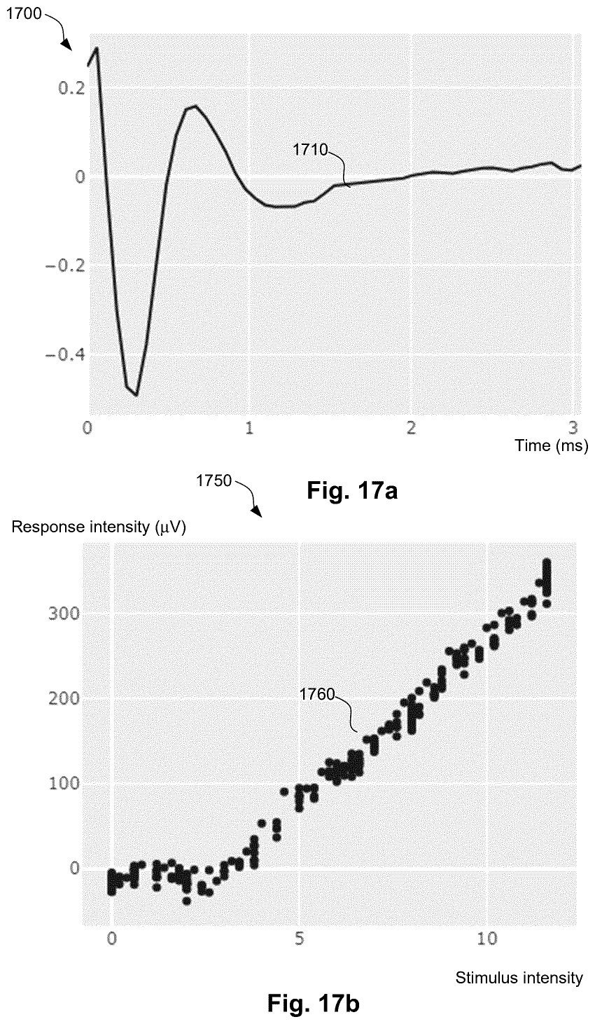

FIG. 17a is a graph 1700 on which is plotted the third basis function 1630 from FIG. 16a, transformed by rotation into the basis function 1710. The rotation is by 32° around the x-axis, 0° around the y-axis, and 49° around the z-axis. FIG. 17b is a graph 1750 containing the resulting activation plot 1760, obtained by correlating the rotated basis function 1710 with the m columns of the data matrix X, i.e. the subset of signals illustrated in FIG. 15. It may be seen that the AP 1760 more closely resembles the piecewise-linear AP model illustrated in FIG. 4a than any of the AP models in FIG. 16.

In a second implementation of step 1420, the candidate detectors are quadrature filters of variable frequency. That is, the candidate detectors are complex-valued vectors u(f) of the form

$$e \{ u ( f ) \} = cos ( 2 π fn ) \qquad ( 8 )$$

$$m \{ u ( f ) \} = sin ( 2 π fn ) \qquad ( 9 )$$

where n is a vector of time instants (integer multiples of the sampling interval in seconds) over which the signals in the data matrix are defined, and f is a frequency in Hz. The frequency f may be varied in discrete steps over a predetermined range, for example [10 Hz, 2 kHz] to derive the set of candidate detectors according to the second implementation of step 1420.

In other implementations of step 1420, other dimensionality reduction analysis methods may be used to derive the candidate detectors, including:
Independent component analysis
T-distributed stochastic neighbour embedding (t-SNE)
Linear discriminant analysis

Returning to the method 1400, step 1430 selects the next candidate detector in the set of candidate detectors constructed at step 1420. Step 1440 then correlates the candidate detector with all the m signals in the subset to obtain a set of m (stimulus intensity(s), response intensity (d)) pairs corresponding to the candidate detector.

In the first implementation with p=3, the correlations with candidate detectors U₃T(θ) derived from non-zero angle vectors θ may be efficiently calculated by post-multiplying the matrix of correlations with the first three candidate vectors (the columns of U₃T(0)) by the rotation matrix T(θ).

If the candidate filters are derived as quadrature filters using the second implementation of step 1410, correlation of a quadrature filter with a signal as in step 1440 comprises correlating each of the real and imaginary parts of the quadrature filter with the signal, and then taking the square root of the sum of the squares of the two correlations.

The next step 1450 fits an activation plot (AP) model to the set of (s, d) pairs obtained at step 1440. In implementations in which the AP model is the piecewise linear model of equation (1), step 1450 fits a piecewise linear AP model to the set of (s, d) pairs in conventional fashion.

In implementations in which the AP model is the GGC model of equation (2), step 1450 fits a GGC model to the set of (s, d) pairs. In such implementations, the nonlinear GGC parameters s_{T}, τ₁, τ₂, and r may be initialised to sensible starting points s_{T0}, τ₀, τ₀, and r₀. In one implementation, these values may be set to:
s_{T0}: the maximum stimulus intensity parameter value in the set of (s, d) pairs
τ₀: between 0.1 and 0.4
r₀: between 3 and 4

(The slope parameter P, being linear, does not need a starting point.) A fitting algorithm such as Trust Region Reflective (TRF) may then be used to optimise the values of the nonlinear parameters s_{T}, τ₁, τ₂, and r from the starting points s_{T0}, τ₀, and r₀. Iterations of TRF to optimise the nonlinear parameters s_{T}, τ₁, τ₂, and r may be interleaved with iterations of ordinary least squares to find the optimal linear parameter P (which, being linear, does not need a starting point) corresponding to the current values of the nonlinear parameters s_{T}, τ₁, τ₂, and r.

Step 1460 then derives a metric M for the current candidate detector from the fitted AP. The metric M is derived from two sub-metrics: a controllability metric M_{c}, and a physiologicality metric Mₚ.

In some implementations, the controllability metric M_{c} is measure of the “goodness of fit” of the AP model to the set of (s, d) pairs. Controllability, so defined, is an indicator of the usefulness of the current candidate detector to provide a feedback variable for a feedback controller of a CLNS system. In one such implementation, the controllability metric M_{c} may be determined as a growth curve quality index (GCQI) for the fitted AP model. The GCQI indicates a signal-to-noise ratio (SNR) of the fitted GGC. Step 1460 may calculate the GCQI by dividing the peak-to-peak amplitude of the fitted AP model by the standard deviation of the residuals of the fitted AP model. The peak-to-peak amplitude of the fitted AP model may be determined as the difference between the response intensities at the extremes of the therapeutic range between the ECAP threshold and the discomfort threshold.

Step 1460 then determines the physiologicality metric Mₚ from the fitted AP. In some implementations, the current candidate detector will receive a high value of the physiologicality metric Mₚ to the extent that the fitted AP matches the patient's subjective perceptual markers, such as perception threshold and discomfort threshold, as recorded during the capturing of the ensemble at step 1410.

The fitted AP may be used to form estimates \tilde{s}ₚ and \tilde{s}_{d} of the patient's perception threshold sₚ and discomfort threshold s_{d}. In one such implementation, the ECAP threshold T is first obtained from the fitted AP. One or more linear predictive models may then be used to derive the perception threshold and discomfort threshold estimates \tilde{s}ₚ and \tilde{s}_{d} from the ECAP threshold T. One example of a linear predictive model relating the ECAP threshold T to the discomfort threshold estimate s_{d} is:

$$T = m \cdot {s}_{d} \qquad ( 10 )$$

where m is a correlation parameter that may be derived from historical patient data. In one implementation, m takes a value between 0.5 and 1.0. In another implementation, m takes a value between 0.6 and 0.9. In one implementation, m takes a value between 0.65 and 0.8.

In another such implementation, suitable for the GGC AP model, the ECAP threshold T, which in one implementation is equal to the intercept s_{T}, and the saturation threshold, which in one implementation is equal to the saturation ratio r times s_{T}, may first be obtained from the fitted AP. One or more linear predictive models may then be used to derive the perception threshold and discomfort threshold estimates \tilde{s}ₚ and \tilde{s}_{d} from the ECAP threshold T and the saturation threshold rs_{T}.

The physiologicality metric Mₚ may then be determined from the norm of the difference d between two vectors: the recorded threshold vector [sₚ s_{d}] and the estimated threshold vector [\tilde{S}ₚ \tilde{S}_{d}]. In one example, the physiologicality metric Mₚ may be determined as

$${M}_{p} = f ( ‖ d ‖ ) \qquad ( 11 )$$

where ƒ is a function defined such that as the norm rises, the physiologicality metric Mₚ decreases. One example of a suitable function ƒ is a decaying exponential ƒ(x)=e^{-kx} where k is a scaling constant. Another example is of a suitable function ƒ is a reciprocal function

$$f ( x ) = \frac{1}{1 + kx} .$$

Step 1460 then determines the overall metric M for the current candidate detector as some combination of the controllability metric M_{c} and the physiologicality metric Mₚ. The combination may be a weighted sum, in which the weights have been previously determined from manually chosen candidate detectors.

Step 1470 then checks whether there are any further candidate detectors for which to derive a metric. If so (“Y”), the method 1400 returns to step 1430 to select the next candidate detector. If not (“N”), step 1480 selects the candidate detector with the highest value of the metric M.

Optionally, a further step 1490 (shown dashed in FIG. 14) assesses the selected detector using the signals from the ensemble recorded at recording electrodes different from the recording electrode corresponding to the subset extracted at step 1420. In one implementation of step 1490, the steps 1420 to 1480 are repeated at each other recording electrode to derive a detector for each recording electrode. If the detectors at each recording electrode are truly measuring a therapeutic component comprising a propagating ECAP from some specific fibre type, it is reasonable to expect that the morphology of the detector would stay roughly constant at different recording electrodes, with the position of its peaks merely shifting as the recording electrode varies along the lead.

This constancy of morphology may be quantified by performing normalised cross-correlations of all the derived detectors with each other and storing the maximum cross-correlation value of each pair of detectors in a cross-correlation matrix. The uniformity of the cross-correlation matrix (i.e. its similarity to a matrix of all ones) is a measure of the constancy of morphology of the detectors across recording electrodes.

Alternative implementations of the method 1400 that take into account all the signals in the ensemble when selecting the optimal candidate detector omit the final step 1490. Instead, such implementations embed all the signals in the ensemble in the controllability and physiologicality metrics for each candidate detector. In such implementations, the conduction velocity of the expected therapeutic response component and the inter-electrode distance along the electrode array 150 are assumed to be known. An expected delay between recording electrodes is predetermined as the inter-electrode distance divided by the conduction velocity. At step 1440, the candidate detector is correlated with all the signals in the subset corresponding to the predetermined recording electrode to obtain a set of (s, d) pairs corresponding to the predetermined recording electrode. This step is repeated for the candidate detector, delayed by an appropriate multiple of the predetermined delay, on each other subset of the ensemble corresponding to a different recording electrode, to obtain a set of (s, d) pairs corresponding to each recording electrode. Step 1450 then fits an AP model to each set of (s, d) pairs obtained at step 1440. Step 1460 then derives an overall metric from the ensemble of fitted APs, by deriving an overall controllability metric as a representative value of the ensemble of individual controllability metrics derived from the individual APs, and an overall physiologicality metric as a representative value of the ensemble of individual physiologicality metrics derived from the individual APs. If the delayed detector at each recording electrode is truly measuring the expected therapeutic component comprising an ECAP propagating at the known conduction velocity, the ensemble of APs will fit the AP model and match the patient's perceptual markers well enough to provide a good overall metric.

In a further optional step (not shown), the derived detector may be applied to signals captured at the corresponding recording electrode over a range of stimulus intensities with the patient in different postures to generate an AP in each subsequent posture. A metric M for each posture may be derived from such APs as described above. The detector derived by the method 1400 in the first posture that maintains a controllable and physiological AP across postures, as reflected in the metric M, is likely to reflect an evoked therapeutic component from some specific fibre type.

Methods and systems according to another aspect of the present technology are configured to derive a plurality of customised vector detectors from the ensemble of captured signals. Such methods and systems are suitable, for example, for a scenario in which the mixed response is expected to contain multiple components, and the intensity of at least one response component is to be estimated. Heuristically, the number (n) of customised vector detectors to be derived according to this aspect is equal to the number of expected response components, plus one (to account for artefact).

In one “sequential” implementation according to this aspect, a variant of the method 1400 may be iterated n times. The variant is the same as the method 1400, except that the physiologicality metric Mₚ is derived in at most one iteration. In other iterations, the metric M derived at step 1460 comprises only the controllability metric M_{c}. Each iteration of the variant method 1400 is followed by a “scrubbing” step (not shown) in which the component corresponding to the selected candidate detector from step 1480 is subtracted from each signal in the ensemble. The corresponding component may be obtained by multiplying the selected detector by the response intensity d from step 1440 corresponding to the selected detector. The next iteration of the method 1400 takes place on the “scrubbed” ensemble.

In other, “parallel” implementations according to this aspect, an n-tuple of candidate detectors is selected from the set of candidate detectors in a single iteration of a method that computes a joint metric M for each candidate n-tuple and then selects the candidate n-tuple with the highest joint metric M. In such parallel implementations, it is convenient to choose p (the size of the kernel of candidate detectors) equal to n (the number of detectors to be selected), since it can then be guaranteed that the n candidate detectors in each candidate n-tuple are orthogonal to each other, which simplifies the calculation of the response intensity d obtained by each candidate detector in an n-tuple.

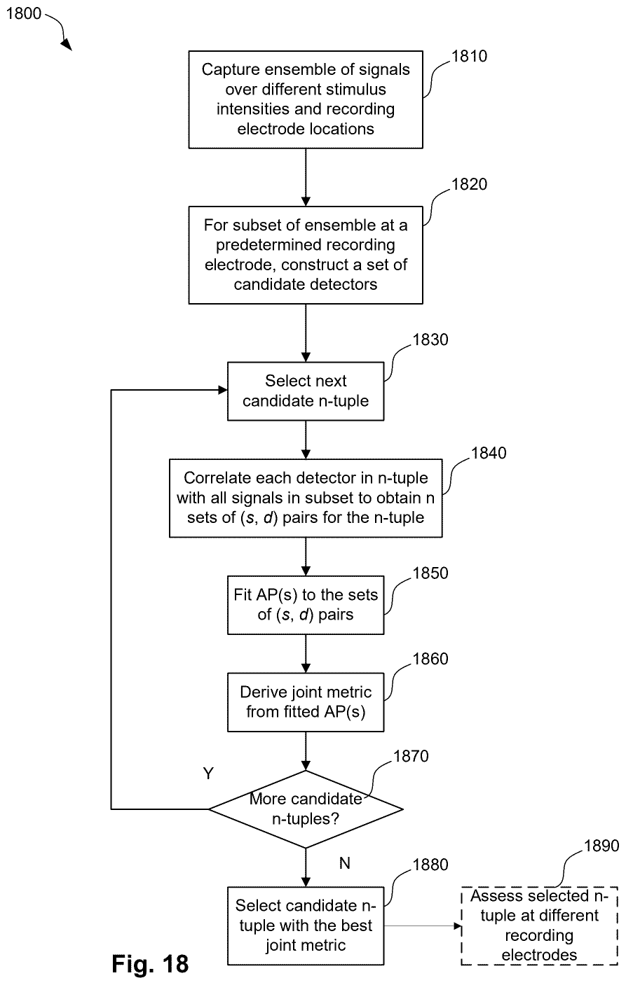

FIG. 18 is a flow chart illustrating a method 1800 of selecting an n-tuple of candidate detectors according to one parallel implementation according to this aspect. The method 1800 is similar to the method 1400, with like numbers indicating like steps (e.g. step 1810 is the same as step 1410), except as described below. Step 1830 selects a given candidate n-tuple of detectors, rather than a single detector. Step 1840 correlates each detector in the n-tuple with all the signals in the subset to obtain, for each stimulus intensity s, a set of n response intensities d corresponding to each of the n detectors in the candidate n-tuple. These may be represented as n sets of (s, d) pairs for the candidate n-tuple. Step 1850 then applies an intensity-combining function to the set of n response intensities d for each stimulus intensity s to obtain a combined response intensity D for s. Step 1850 then fits a single AP to the set of (s, D) pairs for the candidate n-tuple. Step 1870 checks whether there are any remaining candidate n-tuples. Step 1880 selects the candidate n-tuple with the highest value of the joint metric M.

In some such parallel implementations, the intensity-combining function is a weighted sum of the response intensities. In other such parallel implementations, the intensity-combining function is something other than a weighted sum of the response intensities, i.e. some kind of nonlinear combination thereof that is meaningful as a feedback variable for a feedback loop. One example of a nonlinear intensity-combining function on the set of n response intensities is the ratio of the first and second response intensities. Such a combined response intensity D when used as a feedback variable allows a loop to maintain the two corresponding components at or near a target ratio of intensities.

Another example of an intensity-combining function is to “scrub” the components corresponding to each of the n candidate detectors in the n-tuple from the input signal to get a residual signal, then determine the combined response intensity D as the root mean square (RMS) value of the residual signal. Such a combined response intensity D when used as a feedback variable allows a loop to maintain whatever portion of the response is orthogonal to the n detectors at or near a target response intensity. In other words, the n detectors represent components that are to be specifically ignored when computing the feedback variable.

Other examples of deriving a single response intensity D from the set of n response intensities for the candidate n-tuple may be found in International Patent Publication no. WO2024/036380, the contents of which are herein incorporated by reference. This publication describes many implementations of deriving a single feedback variable from multiple response intensities from respective detectors.

In other parallel implementations, step 1850 does not apply an intensity-combining function to the n response intensities d to derive a combined response intensity D at each s value for the candidate n-tuple. Instead, step 1850 fits n APs to the n sets of (s, d) pairs for the candidate n-tuple. Step 1860 then derives a set {M_{c}} of n controllability metrics from the n APs for the candidate n-tuple (for example using the GCQI as described above in relation to step 1460). Step 1860 then applies a metric-combining function to the set {M_{c}} of n controllability metrics to obtain a joint controllability metric M_{c} for the candidate n-tuple. One example of a metric-combining function is a sum of the squared individual metrics.

Step 1860 also derives a joint physiologicality metric Mₚ from the n APs for the candidate n-tuple. In one such implementation, an equation similar to Equation (11) may be used, with the modification that the function/provides a relatively high value when exactly one of the APs matches the patient's sensation and a lower value if more than one of the n APs matches the patient's sensation. In another such implementation, multiple patient sensations are defined, such as paresthesia and muscle activation. Perceptual markers are obtained from the patient for each defined sensation and matched to the n APs for the n-tuple to obtain multiple physiologicality metrics {Mₚ} for the n-tuple using equation (11). These physiologicality metrics {Mₚ} are then combined using a metric-combining function to obtain a joint physiologicality metric Mₚ for the n-tuple.

In some circumstances, the mixed response signal may comprise only artefact and a single evoked response component such as an ECAP. Variants of the method 1800 and 1400 may be utilised to derive a detector for the evoked response component that is optimally insensitive to artefact.

According to one such variant of the method 1800, step 1810 captures only signals from stimulus intensities that are known to be sub-threshold, so that the signals contain only artefact. Step 1850 fits the APs using a purely linear model of artefact dependence on stimulus intensity, i.e. the model of equation (1) with the threshold T set to zero. Step 1860 then determines only the joint controllability metric M_{c} for the candidate n-tuple, since physiologicality is irrelevant to artefact modelling. Step 1880 selects the candidate n-tuple with the highest joint controllability metric M_{c}. The resulting n-tuple, saved in the matrix Uₙ^{a}, is the optimal set of n artefact basis functions for the sub-threshold data set.

A detector for the evoked response component may then be derived from a new data matrix of signals containing both artefact and the artefact response component using a variant of the method 1400. According to the variant, after capturing the ensemble at step 1410, and before constructing the set of candidate detectors at step 1420, artefact may be scrubbed from each signal in the ensemble using the set of n artefact basis functions to construct an “artefact-free” ensemble. The remaining steps 1420 to 1480 may be carried out as described above on the artefact-free ensemble to derive the optimally artefact-insensitive detector for the evoked response component that also matches the patient's sensations.

The systems and methods according to the present technology may be used: intra-operatively to guide implantation; as part of the programming of a CLNS device in-clinic; or out of clinic at regular intervals to adapt the detector to lead migration or some other change in circumstances.

### Interpretation

References herein to estimation, determination, comparison and the like are to be understood as referring to an automated process carried out on data by a processor operating to execute a predefined procedure suitable to effect the described estimation, determination or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion.

### Wireless

The invention may be embodied using devices conforming to other network standards and for other applications, including, for example other WLAN standards and other wireless standards such as MICS. Applications that can be accommodated include IEEE 802.11 wireless LANs and links, and wireless Ethernet.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of this document, the term “wired” and its derivatives may be used to describe circuits, devices, systems, methods, techniques, communications channels, etc., that may communicate data through the use of modulated electromagnetic radiation through a solid medium. The term does not imply that the associated devices are coupled by electrically conductive wires.

### Processes

Unless specifically stated otherwise, as apparent from the following discussions, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

### Processor

In a similar manner, the term “processor” may refer to any device or portion of a device that processes electronic data, e.g., from registers and/or memory to transform that electronic data into other electronic data that, e.g., may be stored in registers and/or memory. A “computer” or a “computing device” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. The processing system further may include a memory subsystem including main RAM and/or a static RAM, and/or ROM.

### Computer-Readable Medium

Furthermore, a computer-readable carrier medium may form, or be included in a computer program product. A computer program product can be stored on a computer usable carrier medium, the computer program product comprising a computer readable program means for causing a processor to perform a method as described herein.

### Networked or Multiple Processors

In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

### ADDITIONAL EMBODIMENTS

Thus, one embodiment of each of the methods described herein is in the form of a computer-readable carrier medium carrying a set of instructions, e.g., a computer program that are for execution on one or more processors. Thus, as will be appreciated by those skilled in the art, embodiments of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media.

### Implementation

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

### Means for Carrying out a Method or Function

Furthermore, some of the embodiments are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

### Implementations

Reference throughout this specification to “one implementation” or “an implementation” means that a particular feature, structure or characteristic described in connection with the implementation is included in at least one implementation of the present invention. Thus, appearances of the phrases “in one implementation” or “in an implementation” in various places throughout this specification are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the invention, various features of the invention are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of this invention.

Furthermore, while some implementations described herein include some but not other features included in other implementations, combinations of features of different implementations are meant to be within the scope of the invention, and form different implementations, as would be understood by those in the art. For example, in the following claims, any of the claimed implementations can be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that each unit between two particular units are also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that implementations of the invention may be practiced without these specific details. In other instances, well-known methods, structures and techniques have not been shown in detail in order not to obscure an understanding of this description.

### Terminology

Throughout this specification, the terms “a” and “an” mean “one or more”, unless expressly specified otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” or “one or more of” a list of options is to be understood to mean that the element may be any one of the listed options, or may be any combination of two or more of the listed options.

In this specification the word “or” is to be read inclusively rather than exclusively, except where otherwise indicated.

Neither the title nor any abstract of the present application should be taken as limiting in any way the scope of the claimed invention.

Where the preamble of a claim recites a purpose, benefit or possible use of the claimed invention, it does not limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean that two or more elements are either in direct physical or causal contact, or that two or more elements are not in direct contact with each other yet still co-operate or interact with each other.

It will be appreciated by persons skilled in the art that numerous variations or modifications may be made to the invention as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present invention. The disclosed implementations are, therefore, to be considered in all respects as illustrative and not limiting or restrictive.

Any discussion of documents, acts, materials, devices, articles or the like which has been included in this specification is solely for the purpose of providing a context for the present invention. It is not to be taken as an admission that any or all of these matters form part of the prior art base or were common general knowledge in the field relevant to the present invention as it existed before the priority date of each claim of this application.

The features described in relation to one or more aspects of the present technology are to be understood as applicable to other aspects of the present technology. More generally, combinations of the steps in the method(s) of the present technology or the features of the system(s) or device(s) of the present technology described elsewhere in this specification, including in the claims, are to be understood as falling within the scope of the disclosure of this specification.

### INDUSTRIAL APPLICABILITY

It is apparent from the above that the arrangements described are applicable to the health care industries.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| ground | 130 |
| array | 150 |
| biphasic stimulus pulse | 160 |
| ECAPs | 170 |
| target fibres | 180 |
| communications channel | 190 |
| external computing device | 192 |
| CLNS system | 300 |
| clinical settings controller | 302 |
| target ECAP controller | 304 |
| box | 308 |
| box | 309 |
| controller | 310 |
| box | 311 |
| pulse generator | 312 |
| element | 313 |
| measurement circuitry | 318 |
| signal window | 319 |
| ECAP detector | 320 |
| comparator | 324 |
| gain element | 336 |
| integrator | 338 |
| graph | 400 |
| activation plot | 402 |
| ECAP threshold | 404 |
| discomfort threshold | 408 |
| perception threshold | 410 |
| therapeutic range | 412 |
| graph | 450 |
| golden growth curve | 460 |
| therapeutic range | 475 |
| vertical line | 480 |
| vertical line | 490 |
| ECAP | 600 |
| neural stimulation system | 700 |
| device | 710 |
| remote controller | 720 |
| CST | 730 |
| CI | 740 |
| charger | 750 |
| data flow | 800 |
| neuromodulation device | 804 |
| CPA | 810 |
| clinical data log file | 812 |
| CDV | 814 |
| clinical Data Uploader | 816 |
| database loader | 822 |
| database | 824 |
| data analysis web API | 826 |
| analysis module | 832 |
| signal | 1010 |
| signal | 1020 |
| method | 1100 |
| step | 1110 |
| step | 1120 |
| step | 1130 |
| step | 1140 |
| step | 1150 |
| step | 1160 |
| ensemble | 1200 |
| signal | 1210 |
| graph | 1300 |
| method | 1400 |
| step | 1410 |
| step | 1420 |
| step | 1430 |
| step | 1440 |
| step | 1450 |
| step | 1460 |
| step | 1470 |
| step | 1480 |
| step | 1490 |
| graph | 1500 |
| signal | 1510 |
| graph | 1600 |
| basis function | 1610 |
| basis function | 1620 |
| basis function | 1630 |
| graph | 1650 |
| activation plot | 1660 |
| activation plot | 1670 |
| activation plot | 1680 |
| graph | 1700 |
| basis function | 1710 |
| graph | 1750 |
| activation plot | 1760 |
| method | 1800 |
| step | 1810 |
| step | 1820 |
| step | 1830 |
| step | 1840 |
| step | 1850 |
| step | 1860 |
| step | 1870 |
| step | 1880 |
| step | 1890 |

1. A neural stimulation system comprising:
an implantable device for controllably delivering neural stimuli, the device comprising:
a stimulus source configured to deliver neural stimuli via one or more stimulus electrodes of a plurality of implanted electrodes to neural tissue of a patient in order to evoke neural responses from the neural tissue;
measurement circuitry configured to capture signal windows from signals sensed on the neural tissue subsequent to respective neural stimuli by a recording electrode of the plurality of implanted electrodes; and
a control unit configured to:
control the stimulus source to deliver a neural stimulus according to a stimulus intensity parameter; and
control the measurement circuitry to capture a signal window from a signal sensed on the neural tissue by a recording electrode subsequent to the neural stimulus; and
a processor configured to:
instruct the control unit to control the measurement circuitry to capture a plurality of signal windows from signals sensed on the neural tissue by a first recording electrode subsequent to respective neural stimuli delivered according to respective stimulus intensity parameter values;
repeatedly, for each candidate detector in a set of candidate detectors:
measure, using the candidate detector, intensities of evoked neural responses in the captured signal windows; and
derive a metric for the candidate detector from the measured neural response intensities; and
select a candidate detector for the first recording electrode from the set of candidate detectors based on their respective metrics.

2. The system of claim 1, wherein each signal window represents a complex electrophysiological response of the neural tissue to a corresponding stimulus.

3. The system of claim 1, wherein the processor is configured to derive the metric by:
fitting an activation plot model to the measured neural response intensities; and
deriving the metric from the fitted activation plot model.

4. The system of claim 3, wherein the processor is configured to derive the metric by deriving a controllability metric from the fitted activation plot model, wherein the controllability metric is an indicator of the usefulness of the candidate detector to provide a feedback variable for a feedback controller.

5. The system of claim 4, wherein the processor is configured to derive the metric by deriving a physiologicality metric from the fitted activation plot model, wherein the physiologicality metric is an indicator of closeness of a match between the fitted activation plot model and sensations experienced by the patient in response to the stimuli.

6. The system of claim 5, wherein the processor is configured to derive the metric by combining the controllability metric and the physiologicality metric.

7. The system of claim 1, wherein the processor is further configured to:
instruct the control unit to control the measurement circuitry to capture a further plurality of signal windows from signals sensed on the neural tissue by a further recording electrode subsequent to respective neural stimuli delivered according to respective stimulus intensity parameter values; and
assess the selected candidate detector using the further plurality of signal windows.

8. The system of claim 1, wherein the processor is further configured to:
scrub the plurality of signal windows using the selected candidate detector; and
repeat the repeated measuring and deriving on the scrubbed signal windows, and the selecting to select a further candidate detector for the first recording electrode.

9. The system of claim 1, wherein the candidate detector is one of a candidate n-tuple of detectors from a set of candidate n-tuples, where n is two or more.

10. The system of claim 9, wherein the processor is configured to repeat the repeated measuring and deriving for each detector in the candidate n-tuple.

11. The system of claim 10, wherein the processor is configured to combine the metrics into a combined metric for the candidate n-tuple using a metric combining function.

12. The system of claim 11, wherein the processor is configured to select a candidate n-tuple for the first recording electrode based on their respective combined metrics.

13. The system of claim 1, wherein the processor is further configured to construct the set of candidate detectors from the plurality of signal windows.

14. The system of claim 13, wherein the processor is further configured to construct the set of candidate detectors as a kernel of basis functions for the plurality of signal windows.

15. The system of claim 14, wherein the processor is further configured to augment the set of candidate detectors by applying a set of orthonormal transformations to the kernel of basis functions.

16. The system of claim 1, wherein the set of candidate detectors is a set of quadrature filters of varying frequency.

17. The system of claim 1, wherein the processor is further configured to program the control unit of the implantable device to use the selected candidate detector to measure intensities of evoked neural responses in captured signal windows.

18. An automated method comprising:
delivering a plurality of neural stimuli to neural tissue of a patient in order to evoke neural responses from the neural tissue, the neural stimuli being delivered according to respective stimulus intensity parameter values;
capturing a plurality of signal windows from signals sensed on the neural tissue by a first recording electrode subsequent to respective neural stimuli delivered according to respective stimulus intensity parameter values;
repeatedly, for each candidate detector in a set of candidate detectors:
measuring, using the candidate detector, intensities of evoked neural responses in the captured signal windows; and
deriving a metric for the candidate detector from the measured neural response intensities; and
selecting a candidate detector for the first recording electrode from the set of candidate detectors based on their respective metrics.

19. The method of claim 18, wherein each signal window represents a complex electrophysiological response of the neural tissue to a corresponding stimulus.

20. The method of claim 18, wherein deriving the metric comprises:
fitting an activation plot model to the measured neural response intensities; and
deriving the metric from the fitted activation plot model.

21. The method of claim 20, wherein deriving the metric comprises deriving a controllability metric from the fitted activation plot model, wherein the controllability metric is an indicator of the usefulness of the candidate detector to provide a feedback variable for a feedback controller.

22. The method of claim 21, wherein deriving the metric comprises deriving a physiologicality metric from the fitted activation plot model, wherein the physiologicality metric is an indicator of closeness of a match between the fitted activation plot model and sensations experienced by the patient in response to the stimuli.

23. The method of claim 22, wherein deriving the metric comprises combining the controllability metric and the physiologicality metric.

24. The method of claim 18, further comprising:
capturing a further plurality of signal windows from signals sensed on the neural tissue by a further recording electrode subsequent to respective neural stimuli delivered according to respective stimulus intensity parameter values; and
assessing the selected candidate detector using the further plurality of signal windows.

25. The method of claim 18, further comprising:
scrubbing the plurality of signal windows using the selected candidate detector; and
repeating the repeated measuring and deriving on the scrubbed signal windows, and the selecting to select a further candidate detector for the first recording electrode.

26. The method of claim 18, wherein the candidate detector is one of a candidate n-tuple of detectors from a set of candidate n-tuples, where n is two or more.

27. The method of claim 26, further comprising repeating the repeated measuring and deriving for each detector in the candidate n-tuple.

28. The method of claim 27, further comprising combining the metrics into a combined metric for the candidate n-tuple using a metric combining function.

29. The method of claim 28, further comprising select a candidate n-tuple for the first recording electrode based on their respective combined metrics.

30. The method of claim 18, further comprising constructing the set of candidate detectors from the plurality of signal windows.

31. The method of claim 30, wherein the constructing comprises constructing the set of candidate detectors as a kernel of orthonormal basis functions for the plurality of signal windows.

32. The method of claim 31, further comprising augmenting the set of candidate detectors by applying a set of orthonormal transformations to the kernel of orthonormal basis functions.

33. The method of claim 18, wherein the set of candidate detectors is a set of quadrature filters of varying frequency.

34. The method of claim 18, further comprising programming an implantable device to use the selected candidate detector to measure intensities of evoked neural responses in captured signal windows.
